(19) United States
(12) Patent Application Publication
(10) Pub. No.: US 2014/0276670 A1
(43) Pub. Date: Sep. 18, 2014

(54) SYSTEM AND METHOD FOR CONTROLLING THE FOCAL POINT LOCATIONS OF A LASER BEAM

(71) Applicant: Technolas Perfect Vision GmbH, Muenchen (DE)

(72) Inventors: Holger Schlueter, Muenchen (DE); Roland Toennies, Gernlinden (DE); Gwillem Mosedale, Muenchen (DE)

(73) Assignee: Technolas Perfect Vision GmbH, Muenchen (DE)

(21) Appl. No.: 13/835,090

(22) Filed: Mar. 15, 2013

(51) Int. Cl. A61F 9/008

(52) U.S. Cl. CPC A61F 9/008
USPC 606/4

(57) ABSTRACT

A system and method are provided for establishing precise locations for a focal point of a laser beam within a predetermined scanning range during ophthalmic laser surgery. An important aspect of the present invention is the use of a tolerance for deviation of the laser beam's focal point from the laser beam path. The purpose of the tolerance is to ensure that the surgical procedure is effective and that collateral damage to non-targeted tissue does not occur. The present invention accounts for deviations caused by various factors during a procedure. A computer is provided to ensure that the cumulative effect of all deviations maintains the focal point within the tolerance.

### FIELD OF THE INVENTION

The present invention pertains generally to systems and methods for performing ophthalmic laser surgery. More particularly, the present invention pertains to systems and methods for performing an ophthalmic laser surgical procedure that accounts for optical distortions introduced by the anatomy of the eye and by the system components that are required for performing laser surgery. The present invention is particularly, but not exclusively, useful as a system and method for establishing precise locations for a focal point within a predetermined tolerance that is established for a scanning range which is required to conduct a particular ophthalmic laser procedure.

### BACKGROUND OF THE INVENTION

Femtosecond laser technology has been adapted for use with various ophthalmic laser surgical procedures. Until recently, the use of femtosecond lasers on the eye has focused primarily on the cornea. As the use of femtosecond lasers becomes more advanced, other areas of the eye that lie beyond the cornea are now being targeted by procedures that use femtosecond lasers. As appreciated by skilled artisans, often laser systems such as picosecond and UV lasers can be similarly employed. In the event when areas beyond the cornea are targeted, establishing precise locations for the focal point of the laser becomes less precise due to deviations in focal point location caused by: (1) the location in the eye where the procedure is being conducted; (2) system components required for the procedure; and (3) the interaction of the system with the eye during the procedure. Furthermore, these deviations in focal point location can cause the laser procedure to be less effective, or cause damage to areas of the eye that are not being targeted by the procedure.

For any laser surgical procedure, attention must be paid to deviations in focal point location. Different surgical procedures target different areas of the eye, and targeted areas of the eye may be more prone to deviations because of factors like depth within the eye or the anatomical structure of the targeted area. Deviations can also occur based on distortions of the eye that occur due to contact of the eye by the laser unit during the procedure.

System components required for a laser surgical procedure can also produce deviations in focal point location. One important component required by a laser system for positioning a laser beam focal point is an algorithm that is used by a computer to produce a reference datum. Such a reference datum is needed for accurate movement and placement of the focal point during the procedure. Typically, each algorithm will introduce a deviation because of the level of detail it provides for the reference datum. This will vary depending on the algorithm that is selected. Another component that can produce deviations is the lens or lenses in the optical unit. In particular, during a procedure, the lens or lenses must follow a very precise path to accurately focus the laser to a focal point. Further, imprecise or inaccurate mechanical responses of the laser system (e.g. inaccurate lens movement) will also introduce deviations in focal point location. Other deviations in focal point location can be caused by distortions in the eye which are caused when a patient interface is used to facilitate a connection between the laser unit and the eye.

One way to account for the deviations that may be caused by the factors discussed above is to establish a tolerance. In the context of the present invention, the tolerance is an acceptable margin for error in the location of the focal point during a procedure. This tolerance is selected to allow for slight deviations to occur that will not affect the quality of the laser procedure being performed or damage non-targeted areas of the eye.

In light of the above, it is an object of the present invention to provide systems and methods for establishing precise locations for a focal point of a laser. Another object of the present invention is to provide systems and methods for establishing precise locations for a focal point that accounts for deviations caused by the interaction of a laser system with the eye. Still another object of the present invention is to provide systems and methods establishing precise locations for a focal point of a laser beam within a predetermined scanning range that are easy to use and comparatively cost effective.

### SUMMARY OF THE INVENTION

In accordance with the present invention, a system and method are provided for precisely positioning the focal point of a laser beam during laser ophthalmic surgery. Initially, the particular surgical procedure to be performed is selected and identified. For the present invention it is envisioned that such a procedure may be performed anywhere in an eye where a laser beam can be effectively focused (examples include, but are not limited to, the cornea, the crystalline lens, the trabecular meshwork, and the retina). Based on the requirements of the selected procedure, a tolerance for deviation of the laser beam's focal point from the laser beam path during the procedure is determined and established. For instance, it may be necessary to keep the laser beam's focal point beyond a certain distance from an identified tissue interface. Specifically, this restriction may be necessary in order to prevent unwanted collateral damage to a non-targeted tissue, or because the application demands high precision. In each case, as implied above, such considerations go to establishing the tolerance for the particular procedure.

It is understood by the present invention that various factors can affect the operation of a laser system and, consequently the placement of its laser beam's focal point. Of particular concern here is the extent to which these factors, individually and collectively, will cause the focal point to deviate from an intended beam path during an operational procedure. In the event, whatever deviations in focal point placement may be introduced, their cumulative effect must not exceed the limitations of the predetermined tolerance. For the present invention, the factors which can create focal point deviations that are of particular interest include: 1) the mechanical response of the optical components in a laser system during the focusing and placement of a laser beam's focal point; 2) the accuracy of the algorithm that is to be used by a laser system for operational guidance and control of a laser beam's focal point; and 3) distortions of the eye that may be caused when a patient interface is used to bring a laser unit into contact alignment with an eye.

Structurally, the system of the present invention includes a laser unit for generating a laser beam, and it includes a moveable lens that is mounted on a rail for focusing the laser beam to a focal point. Also included is a computer for defining a path for movement of the focal point in an x-y-z space during surgery. Specifically, movement of the focal point will be within the scanning range that is required by the surgical procedures and, most importantly, the position of the focal point will be confined to within an established tolerance.

In order to minimize deviations in out-of-tolerance focal point movements that may be introduced by the laser unit itself, an arrestor is selectively positioned on the rail to fix a start position for the lens. An actuator then moves the lens along the rail from the start position in response to instructions from the controller. In particular, the start position is selected to keep required lens movements close to the start position. This is done to thereby minimize out-of-tolerance deviations that may otherwise be caused when lens movements are farther from the start position and less controllable. As envisioned for the present invention, additional arrestors can be positioned along the rail, as desired, to minimize focal point deviations in selected areas of the scanning range.

Another structural component of interest for the present invention is an optical imaging device that can be used to produce an image of the x-y-z space required by the ophthalmic procedure. Once created, the image is inputted into an algorithm to establish a reference datum for movement of the focal point along a defined path in the x-y-z space. As is well known, different types of imaging devices, and different algorithms have different levels of accuracy and precision. With this in mind, the present invention requires selection of an algorithm that establishes a reference datum which will effectively maintain precise locations for the focal point within the predetermined tolerance. Preferably, the ophthalmic imaging device for the present invention will be an Optical Coherence Tomography (OCT) scanner or a Hartmann-Shack sensor. In addition to an OCT scanner and a Hartmann-Shack sensor, other types of imaging devices appropriate for use with the present invention include: a topographic imaging unit, a Scheimpflug imaging unit, a confocal imaging unit, a two-photon imaging unit, a laser range finding imaging unit, and a non-optical imaging unit.

Still another structural component that is often used in a laser system which may cause a laser focal point to deviate from its intended focal point is a patient interface. Specifically, patient interfaces are sometimes used to establish an interaction between the laser unit and an eye of a patient that will stabilize the eye during a surgical procedure. In use, however, patient interfaces can distort the eye and thereby introduce deviations in focal point placements. Typical patient interfaces, in order of improving operational effect (i.e. less distortion), include an applanation lens, a concave lens, and a water-filled lens. Like the other structural components mentioned above, the patient interface needs to be selected to maintain the focal point within the predetermined tolerance.

In an alternate embodiment, instead of using a single lens for positioning the focal point of the laser beam along the z-axis, two lenses are used. More specifically, the system can include two lenses mounted for movement on the rail, with a first (proximal) lens mounted on the rail nearer to the laser unit and a second (distal) lens mounted on the rail further from the laser unit. For this embodiment, the distal lens can be selectively positioned at one of a plurality of distal start points. Specifically, each of these distal start points corresponds to a particular ophthalmic procedure. For example, when the distal lens is positioned at a selected start point, the system will be configured for moving the laser beam's focal point to treat tissue in a specific portion of the eye (e.g. cornea, crystalline lens or retina). Preferably, throughout the procedure, the distal lens remains stationary and the proximal lens moves along the rail. This structural cooperation moves the focal point of the laser beam in the manner required for the present invention.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic of the components of a system in accordance with the present invention;

FIG. 2A is an illustration of the relationship of the scanning range, the tolerance, and z-value for the present invention;

FIG. 2B is an illustration of the cumulative effect of deviations caused by various factors;

FIG. 3 is a flowchart of the operation of the present invention;

FIG. 4 is a schematic of an alternate embodiment of components of a system in accordance with the present invention when two lenses are used; and

FIG. 5 is a diagram of the effect a second lens can have on different scanning ranges.

### DESCRIPTION OF THE PREFERRED EMBODIMENTS

Referring initially to FIG. 1, an ophthalmic laser system in accordance with the present invention is shown and is generally designated 10. As shown, the system 10 includes a computer 12, an imaging unit 14, and a laser unit 16. Taken together, these components of the system 10 will cooperate with each other to direct a laser beam 18 from the laser unit 16 and toward an eye 20 for the purpose of performing laser surgery on the eye 20.

Various other components illustrated in FIG. 1 are also required for the present invention. It can be seen that laser beam 18 is directed from the laser unit 16 to a lens 22 that is mounted on a rail 24. After passing through the lens 22, the beam 18 is directed to a focal point 26 in the eye 20. As shown, the lens 22 is affixed to a sled 28 via a connecting rod 30 for movement during laser surgery. For movement of the lens 22 along the rail 24, an actuator 32 is provided that is electronically connected between the lens 22 and the computer 12, which provides movement instructions to the actuator 32. Two mechanical stops, an arrestor 34 and a second arrestor 36, are also provided to limit the movement of the lens 22 along the rail 24. In addition to limiting movement in this way, the arrestor 34 and the second arrestor 36 may also be used as calibrated reference points for movement of the lens 22. This is accomplished by moving the lens 22 into a position where the sled 28 contacts the arrestor 34 or the second arrestor 36 prior to the start of, or any time during, a procedure. Thus, for some types of procedures, the calibrated reference point is also a start point for the procedure. Moreover, for some types of procedures, such as procedures that do no require the accuracy afforded by a calibrated reference point, it may not be required to move the lens 22 against the arrestor 34 or 36 at the start of, or during, the procedure. The arrestor 34 is a shaft formed onto the laser unit 16 and extending in a horizontal direction away from the laser unit 16. For the second arrestor 36, it is envisioned to be a wheel stop with a shape like the one shown in FIG. 1. Or, the second arrestor 36 can also take the shape of the arrestor 34. At any point during the procedure, the distance of the lens 22 from the arrestor 34 can be measured to have a value of “L” 38. This measurement is accomplished by the interaction of a sensor (not shown) connected to the lens 22 or sled 28 that reads a plurality of incremental reference lines formed onto the rail 24.

In FIG. 1, a patient interface 40 is also shown in contact with the eye 20. As described earlier, the patient interface 40 can be any type appropriate for use for the particular procedure. Three types of patient interface 40 that are suitable for use with the present invention are: an applanation lens, a concave lens, and a water-filled lens. When choosing a particular patient interface 40, an operator will consider the type of procedure being performed, as well as the effect of the lens 22 on the anatomical structure of the eye 20 of a patient.

Now referring to FIG. 2A, the relationship between a scanning range 42 and tolerance 44 is shown. It can be seen that the scanning range 42 includes a start point 46. The lens 22 begins at the start point 46 and moves in a z-direction along the z-axis 48 to focus the laser beam 18 to locations on a defined optical path. In FIG. 2A, two exemplary locations for the focal point 26 on the defined path are shown and generally designated 50a and 50b. In FIG. 2A, location 50a is closer to the start point 46, which means location 50a is closer to the laser unit 16 than location 50b. In order to locate the focal point 26 at location 50a and 50b during a procedure, the computer 12 instructs the actuator 32 to move the lens 22 to a particular position on the rail 24 that will focus the laser beam 18 at the appropriate location 50a, 50b. For the exemplary scanning range 42 shown in FIG. 2A, the tolerance 44 is the same for both locations 50a and 50b. This is because the selected tolerance 44 always remains substantially the same throughout any selected scanning range 42. Different tolerances 44 are only used when multiple scanning ranges are used during a procedure, which is not the case illustrated in FIG. 2A.

Again referring to FIG. 2A, each location 50a, 50b has an associated deviation distance 52, with location 50a having a deviation distance 52a, and location 50b having a deviation distance 52b. These deviation distances 52a, 52b represent total deviations that account for deviations caused by any factor. As shown, deviation distance 52b has a larger magnitude than deviation distance 52a. This occurs because the lens 22 is further from the start point 46. And, the further the lens 22 is moved from the start point 46, the less will be the accuracy of the focal point position of the laser beam 18, and the greater will be the deviation distance 52. Despite the difference in magnitude, both deviation distances 52a, 52b are within the tolerance 44 for the scanning range 42. Additionally, arrow 56 is provided to show that the focal point 26 for the scanning range 42 can move in any forward and backward along the z-axis 48.

Referring now to FIG. 2B, the cumulative aspect of a total deviation is illustrated. For a selected ophthalmic procedure, several factors induce deviations in the location of the focal point 26 during the procedure. As shown, the cumulative effect of all deviations must still maintain the focal point 26 within the tolerance 44. In FIG. 2B, three deviations are shown: (1) deviation 58 for deviations induced by the selected algorithm; (2) deviation 60 for deviations caused by the inaccurate movement of the lens 22; and (3) deviation 62 for deviations caused by the selected patient interface 40. As illustrated, each of the three deviations 58, 60, 62 has a unique value, and when added together, the sum of the three deviations 58, 60, and 62 is deviation 63. As shown, deviation 63 is less than the tolerance 44 (deviation 58+deviation 60+deviation 62<T).

In FIG. 3, a flowchart is used to demonstrate the operation of the present invention. To begin, a procedure is selected in action block 64. This procedure can be any type of procedure that requires the use of a femtosecond laser, and the procedure can occur at any depth in the eye 20. When a procedure is selected, an associated protocol is also selected that will include, at a minimum, the appropriate scanning range 42 required for the procedure. With the tolerance 44 determined, the computer 12 establishes an optical path for the focal point 26 in action block 68. Once the optical path has been determined, the computer 12 conducts an analysis to ensure that the focal point 26 remains within the tolerance 44 due to deviations induced by the path as shown in inquiry block 70. If the focal point 26 is within the tolerance 44, an algorithm is selected in action block 72. If the focal point 26 is not within the tolerance 44, the computer 12 calculates whether the focal point 26 can be brought into tolerance 44 at inquiry block 74. If the focal point 26 can be brought into tolerance 44, then the actuator 32 is adjusted to incorporate a new optical path at action block 76. If the computer 12 determines that the focal point 26 cannot be brought into tolerance 44 at inquiry block 74, the decision is made whether to restart the procedure at inquiry block 78. When the procedure is restarted, the system 10 is reconciled at action block 80 and a new tolerance 44 is determined at action block 66. If a decision is made at inquiry block 78 to not restart the procedure, then the procedure is stopped at action block 82.

Continuing the procedure after an optical path has been determined to be within the tolerance 44, an algorithm is selected at action block 72. This algorithm is used to produce a reference datum that is used to guide the focal point 26 during the procedure. It will be appreciated that, if two or three positions on a corneal surface are measured, only second-order Zernike polynomial coefficients can be accurately calculated. That is, the spherical shape or the cylindrical shape can be determined. If ten points on a corneal surface are measured, then third-order Zernike polynomial coefficients can be calculated. If fifteen points on a corneal surface are measured, then fourth-order Zernike polynomial coefficients can be calculated. That is, defocus, spherical aberration, second order astigmatism, coma, and trefoil can be calculated. After the algorithm is selected, the computer 12 then determines whether the focal point 26 is within the tolerance 44 due to deviations induced by the algorithm at inquiry block 84. It should be noted that the computer 12 in inquiry block 84 also must account for deviations caused by the optical path (See action block 68). If the focal point is within the tolerance 44, the planning of the procedure continues. If it is not, the computer 12 again determines whether the focal point 26 can be brought into tolerance 44, and if it can be brought into tolerance 44 at inquiry block 86, the algorithm is modified at action block 88. The decision to either restart or stop the procedure is the same as described earlier with inquiry block 78 and action blocks 80 and 82.

Once it has been determined that the focal point is in tolerance at inquiry block 84, a patient interface 40 is selected or detected at action block 90. Once the patient interface 40 is selected or detected, the computer 12 determines whether the focal point 26 remains within the tolerance 44 due to deviations caused by the patient interface 40 at inquiry block 92. At inquiry block 92, the computer 12 is still accounting for deviations caused by the optical path and the algorithm (See blocks 68 and 84). If the focal point 26 is within the tolerance 44, then the procedure is conducted as depicted in action block 94. If the focal point 26 is not within the tolerance 44, the computer 12 again determines whether it can be brought within the tolerance 44 at inquiry block 96. If the focal point 26 can be brought within the tolerance 44, then a new patient interface 40 is selected at action block 98. If the focal point 26 cannot be brought into tolerance 44 at inquiry block 96, a decision is again made at block 78 to restart or stop the procedure. The follow-on steps to inquiry block 78 are the same as disclosed previously.

Referring now to FIG. 4, an alternate embodiment for the system of the present invention is shown and is generally designated 100. As shown, in addition to the components disclosed above for the system 10, the system 100 includes an additional lens 102. More specifically, the lens 102 is mounted on a sled 104 for coaxial movement on rail 24, relative to the lens 22. Movement of the lens 102 on rail 24 is provided by an actuator 106 that interconnects the lens 102 with the computer 12. In this arrangement, the lens 22 is sometimes hereinafter referred to as the “proximal lens 22” and the lens 102 will then be referred to as the “distal lens 102”.

FIG. 4 also shows that the lens 102 can be moved, and selectively stopped, at any of three different arrestors (i.e. arrestor 108, arrestor 110 and arrestor 112). As envisioned for the present invention, the arrestors 108, 110 and 112 are positioned in alignment along the rail 24 to establish respective start points for the lens 102. As indicated in FIG. 4, regardless which arrestor (i.e. 108, 110 or 102) may be used with the lens 102, the lens 102 is intended to cooperate in combination with the lens 22 from the selected start point. The significance of this is best appreciated with reference to FIG. 5.

As shown in FIG. 5, when the distal lens 102 is positioned at the arrestor 108, a cooperative interaction of the distal lens 102 with the proximal lens 22 will move the focal point 26 of the laser beam 18 within a scanning range 114. The import here is that the scanning range 114 is effective for ophthalmic procedures which are to be performed in the front (i.e. cornea) of the eye 20. Similarly, when the distal lens 102 is positioned at the arrestor 110 (e.g. lens 102′), the cooperative interaction of the distal lens 102′ with the proximal lens 22 will move the focal point 26′ of the laser beam 18′ within a scanning range 116. In this case, the scanning range 116 needs to be effective for surgeries deeper in the eye 20 (e.g. crystalline lens). Likewise, when the distal lens 102 is positioned at the arrestor 112 (e.g. lens 102″), the cooperative interaction of the distal lens 102″ with the proximal lens 22 will move the focal point 26″ of the laser beam 18″ in a scanning range 118 for procedures performed deep in the eye 20 (e.g. retina).

For each of the above described scenarios (i.e. respective scanning ranges 114, 116 and 118) it will be appreciated that either the proximal lens 22, or the distal lens 102, can be moved from their respectively selected start points to move focal point 26 within a selected scanning range 114, 116 or 118. In each case, the respective start points for lenses 22 and 102 will be established by an arrestor. Specifically, lens 22 will operate relative to the arrestor 34, and lens 102 will operate relative to whichever arrestor 108, 110 or 112 is to be used for a selected procedure. During an operation of the system 100, however, only one of the lenses, lens 22 or lens 102 will be moved to vary the location of the focal point 26 within the particularly selected scanning range 114, 116 or 118.

While the particular System and Method for Controlling the Focal Point Locations of a Laser Beam as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A device for establishing precise locations for a focal point of a laser beam within a predetermined scanning range which comprises:
a laser unit for generating the laser beam;
a lens for focusing the laser beam to the focal point;
an arrestor selectively mounted on the laser unit for interacting with the lens to establish a calibration reference point for the focal point scanning range;
a computer using an algorithm for defining a path for movement of the focal point in an x-y-z space within the predetermined scanning range; and
an actuator for moving the lens from a start point in response to instructions from the computer, and to move the focal point of the laser beam in a z-direction along the defined path in the scanning range.

2. A device as recited in claim 1 wherein the computer determines whether an interaction between the lens and the arrestor is necessary after receiving a user input indicating a particular type of ophthalmic procedure to be performed.

3. A device as recited in claim 1 further comprising a patient interface for establishing an interaction between the laser unit and an eye of a patient, wherein the patient interface is selected to maintain the focal point within the tolerance.

4. A device as recited in claim 3 wherein the patient interface is selected from a group comprising an applanation lens, a concave lens, and a water-filled lens.

5. A device as recited in claim 1 wherein the lens is a proximal lens and the device further comprises:
a distal lens positioned between the proximal lens and an eye of a patient; and
a second actuator for moving the distal lens, in concert with the proximal lens, in response to instructions from the computer to maintain the focal point of the laser beam within the scanning range.

6. A device as recited in claim 1 further comprising an optical imaging device to produce an image of the x-y-z space, wherein the image is inputted into an algorithm to establish a reference datum for movement of the focal point on the defined path in the x-y-z space, and wherein the reference datum maintains establishment of precise locations for the focal point within the tolerance.

7. A device as recited in claim 6 wherein the optical imaging device is selected from a group comprising an Optical Coherence Tomography (OCT) scanner and a Hartmann-Shack sensor, a confocal detector, a Scheimpflug imager, a two-photon imager, a laser range finding imager and a non-optical imager.

8. A device as recited in claim 1 further comprising a rail mounted on the laser unit, wherein the lens is mounted on the rail and the rail is oriented substantially parallel to the laser beam.

9. A device as recited in claim 8 wherein the rail is formed with a plurality of incremental reference lines for indicating a distance “L” from the start position to the lens, and wherein the device further comprises a sensor mounted on the laser unit, wherein the sensor determines a position of the lens on the rail by interacting with the incremental reference lines of the rail.

10. A system for maintaining precise focal point placements during an ophthalmic surgical procedure which comprises:
an imaging unit for creating an image of a selected volume of tissue within an eye of a patient;
a laser unit for generating a laser beam;
a focusing element included in the laser unit for focusing the laser beam to a focal point in the volume of tissue to perform an ophthalmic surgical procedure in accordance with a particular protocol, wherein the protocol requires maintaining the laser beam focal point within a predetermined tolerance; and
a computer for moving the focal point within a predetermined scanning range and along a defined path through the volume of tissue, while maintaining the focal point within the predetermined tolerance.

11. A system as recited in claim 10 wherein the focusing element is moveable from a start point established to locate the focal point in the predetermined scanning range, and wherein deviations of the focal point from the defined path remain within the predetermined tolerance.

12. A system as recited in claim 11 wherein the image is entered into an algorithm to create a reference datum for movement of the focal point along the defined path and wherein the system further comprises a patient interface device for establishing an interaction between the laser unit and an eye of a patient, wherein deviations of the focal point from the defined path caused by the interface device remain within the predetermined tolerance.

13. A system as recited in claim 11 wherein the sum of the deviation caused by the defined path, the deviation caused by the algorithm, and the deviation caused by the interface device is less than the predetermined tolerance.

14. A system as recited in claim 10 wherein the focusing element is a proximal focusing element, and the system further comprises a distal focusing element positioned between the proximal focusing element and the eye.

15. A system as recited in claim 10 wherein the imaging unit is selected from a group comprising a Hartmann-Shack sensor, an Optical Coherence Tomography (OCT) scanner, a topographic imaging unit, a Scheimpflug imaging unit, a confocal imaging unit, a two-photon imaging unit, a laser range finding imaging unit, and a non-optical imaging unit.

16. A method for establishing precise locations for a focal point of a laser beam in a predetermined scanning range, wherein the focal point remains within a predetermined tolerance, the method comprising the steps of:
providing a laser unit to generate a laser beam;
positioning a lens for focusing the laser beam to the focal point;
defining a path for movement of the focal point in an x-y-z space within the predetermined scanning range, wherein the path is defined by a computer;
selectively mounting an arrestor on the laser unit to interact with the lens to establish a start point for the scanning range; and
using an actuator to move the lens in response to instructions from the computer, to move the focal point of the laser beam along the defined path in the predetermined scanning range to maintain the focal point within the tolerance.

17. A method as recited in claim 16 further comprising the steps of:
creating an image of the eye of a patient using an optical imaging device; and
entering the image of the eye into an algorithm to establish a reference datum for movement of the focal point.

18. A method as recited in claim 16 further comprising the step of facilitating the interaction of the laser unit with an eye of a patient with a patient interface device, wherein the patient interface device is selected from a group comprising an applanation lens, a concave lens, and a water-filled lens.

19. A method as recited in claim 18 wherein movement of the lens moves the focal point on the path with a minimal deviation of the focal point within the tolerance, and wherein the algorithm introduces a minimal deviation of the focal point within the tolerance, and wherein the patient interface introduces a minimal deviation of the focal point within the tolerance, and wherein the sum of the deviations caused by the path, the algorithm, and the interface device is less than the tolerance.

20. A method as recited in claim 16 wherein the lens is a proximal lens and wherein the method further comprises the steps of:
providing a distal lens, wherein the distal lens is positioned between the proximal lens and an eye of a patient; and
moving the distal lens in concert with the proximal lens to establish the focal point of the laser beam within the specified scanning range.
